(19) United States
(12) Patent Application Publication
(10) Pub. No.: US 2025/0382259 A1
(43) Pub. Date: Dec. 18, 2025

(54) METHOD FOR PREPARING CHELATABLE EDDHA

(71) Applicant: RAM TECHNOLOGY CO., LTD., Yongin-si (KR)

(72) Inventors: June Ing KIL, Seoul (KR); Jeong Jun Park, Seoul (KR); Young Su Jang, Hwaseong-si (KR)

(73) Assignee: RAM TECHNOLOGY CO., LTD., Yongin-si (KR)

(21) Appl. No.: 19/237,530

(22) Filed: Jun. 13, 2025

(30) Foreign Application Priority Data
Jun. 14, 2024 (KR) 10-2024-0077367

(51) Int. Cl. C07C 227/16; C07C 227/40

(52) U.S. Cl. CPC C07C 227/16; C07C 227/40

(57) ABSTRACT

The present disclosure relates to a method for preparing chelatable EDDHA, and more particularly, to a preparation method capable of preparing high-purity chelatable EDDHA with an excellent yield.

### TECHNICAL FIELD

The present disclosure relates to a method for preparing chelatable EDDHA, and more particularly, to a preparation method capable of preparing high-purity chelatable EDDHA with an excellent yield.

### BACKGROUND ART

EDDHA represented by ethylenediamine-N,N′-bis(2-hydroxyphenylacetic acid is a chelating agent, and one of hexadentate ligands binding to a metal ion using two amines, two phenolate centers and two carboxylates as six binding sites.

As an existing method for synthesizing EDDHA, known is a method of reacting a reaction material including phenol, ethylenediamine, glyoxylic acid and sodium hydroxide at 70° C. to 75° C., and then precipitating a product for 72 hours or longer at room temperature (Structure and fertilizer properties of byproducts formed in the synthesis of EDDHA, Journal of agricultural and food chemistry, 2006, 54, 4355-4363).

However, the reaction requires a precipitation time of at least three days to collect the product after the synthesis, and due to this relatively long process time, it is difficult to produce chelatable EDDHA on an economical and industrial scale, and since the product produced using the method includes a large number of residues and other byproducts, other organic solvents are additionally used to purify the product, causing a problem of generating additional waste liquid.

In addition, chelatable EDDHA has a strong affinity for specific metal ions, and may be used in a substrate surface cleaner, a resister stripper, a slurry for chemical mechanical polishing, an etchant and the like in a semiconductor process, and since a semiconductor process is significantly affected by impurities, the prepared EDDHA needs to be synthesized as a high-purity compound that does not include unnecessary metals in order to be used in the semiconductor process.

Accordingly, development of a new method for preparing EDDHA capable of preparing high-purity chelatable EDDHA with an excellent yield is required.

### DISCLOSURE

### Technical Problem

The present disclosure is directed to providing a preparation method for synthesizing high-purity chelatable EDDHA with an excellent yield.

According to the present disclosure, chelatable EDDHA may be prepared on an economical and industrial scale by synthesizing EDDHA with an excellent yield through synthesizing a mixture of substituted or unsubstituted phenol, ethylenediamine, glyoxylic acid and sodium hydroxide while raising the temperature to a predetermined temperature and thereby applying sufficient heat energy required for the synthesis reaction, and increasing a collection rate of the product through precipitation with heating at a certain temperature.

In addition, chelatable EDDHA prepared using the preparation method has a low metal content in the product, and may be used for various purposes in a semiconductor process.

### Technical Solution

One embodiment of the present disclosure provides a method for preparing chelatable EDDHA, the method including the steps of: mixing substituted or unsubstituted phenol, ethylenediamine, glyoxylic acid and sodium hydroxide (S1); synthesizing a product by reacting the mixture at 75° C. to 80° C. (S2); collecting the product by heating the product to 55° C. to 65° C. (S3); and washing the collected product using a cleaning solution including deionized water (S4).

In step (S2), the mixture may be reacted for 2 hours to 4 hours at 75° C. to 80° C. to synthesize a product.

In step (S3), the product may be heated for 30 minutes to 2 hours at 55° C. to 65° C. to collect the product.

A concentration of the sodium hydroxide may be from 35 wt % to 50 wt %.

A synthesis yield of the product prepared using the preparation method may be 18% or greater.

Amounts of Al and Fe included in the product prepared using the preparation method may each be 5 ppm or less.

Amounts of K and Ca included in the product prepared using the preparation method may each be 5 ppm or less.

An amount of Na included in the product prepared using the preparation method may be 40 ppm or less.

The cleaning solution may further include an additive selected from the group consisting of an organic solvent including acetone, an organic base including NH₄OH, an inorganic acid including HNO₃ and HCl, and combinations thereof.

The cleaning solution may include the deionized water and the additive selected from the group consisting of an organic solvent including acetone, an organic base including NH₄OH, an inorganic acid including HNO₃ and HCl, and combinations thereof in a weight ratio of 5:1 to 20:1.

### Advantageous Effects

When using a method for preparing chelatable EDDHA provided in the present disclosure, high-purity chelatable EDDHA can be prepared with an excellent yield.

In addition, EDDHA prepared using the preparation method has a low metal content in the product, and can be used for various purposes in a semiconductor process.

### BEST MODE

Unless defined otherwise in the present specification, all technical terms and scientific terms have the same meaning as meanings commonly understood by those skilled in the art. Terms used for the description in the present disclosure are only to effectively describe specific embodiments and are not intended to limit the present disclosure.

Singular forms used in the present specification include plural forms as well, unless the context clearly indicates otherwise.

The term ‘include’ used in the present specification specifies specific features, areas, integers, steps, operations, elements and/or components, and does not exclude the presence or addition of other specific features, areas, integers, steps, operations, elements, components and/or groups.

The present disclosure may have various modifications applied thereto, and may have various forms, and specific embodiments will be illustrated and described in detail below. However, this is not intended to limit the present disclosure to specific disclosed forms, and needs to be construed as including all modifications, equivalents and substitutes included in the idea and the technical scope.

In the present specification, when a positional relationship between two parts is described as, for example, ‘˜on’, ‘˜in an upper portion of’, ‘˜in a lower portion of’, ‘˜next to’ and the like, one or more other parts may be located between the two parts unless an expression such as ‘right’ or ‘directly’ is used.

In the present specification, when a temporal relationship is described as, for example, ‘˜after’, ‘˜subsequent to’, ‘˜then’, ‘˜prior to’ and the like, cases that where operations are not continuous may also be included unless an expression such as ‘immediately’ or ‘directly’ is used.

In the present specification, the term ‘at least one’ needs to be construed as including all combinations presentable from one or more related items.

Hereinafter, a method for preparing chelatable EDDHA according to specific embodiments of the present disclosure will be described in more detail.

According to one embodiment of the present disclosure, there is provided a method for preparing chelatable EDDHA, the method including: mixing substituted or unsubstituted phenol, ethylenediamine, glyoxylic acid and sodium hydroxide (S1); synthesizing a product by reacting the mixture at 75° C. to 80° C. (S2); collecting the product by heating the product to 55° C. to 65° C. (S3); and washing the collected product using a cleaning solution including deionized water (S4).

EDDHA may be industrially prepared by a Mannich-like reaction between substituted or unsubstituted phenol, ethylenediamine, glyoxylic acid and sodium hydroxide as shown in the following Reaction Formula 1.

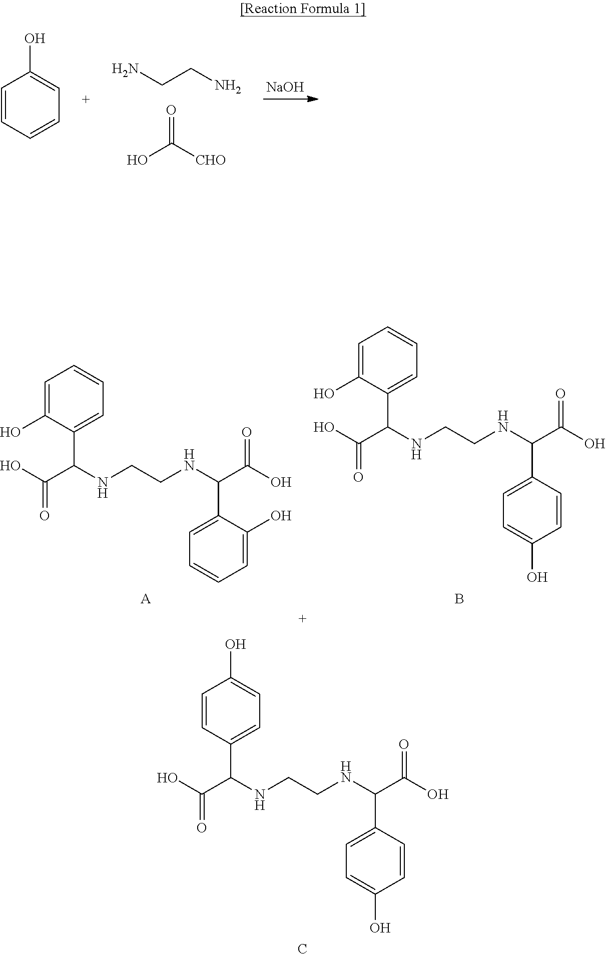

Herein, the synthesis of EDDHA produces A as a main component together with a mixture of position isomers identified as B and C, and other byproducts are produced in various amounts depending on the reaction condition.

A is ethylenediamine-N,N′-bis(2-hydroxyphenylacetic acid, and referred to as o,o-EDDHA, and is chelatable EDDHA capable of forming a hexadentate ligand by binding to a metal ion.

B is 2-((2-((carboxy(2-hydroxyphenyl)methyl)amino)ethyl)amino)-2-(4-hydroxyphenyl)acetic acid, and referred to as o,p-EDDHA, and is capable of chelating with a metal ion by including a structure in which a phenyl group is substituted with a hydroxyl group in an ortho direction.

C is 2,2′-(ethane-1,2-diylbis(azanediyl))bis(2-(4-hydroxyphenyl)acetic acid), and referred to as p,p-EDDHA, and is not able to chelate with a metal ion by having a structure in which all phenyl groups are substituted with a hydroxyl group in a para direction.

In other words, A, B and C are all compounds referred to as EDDHA, however, only A and B are able to chelate with a metal ion, and therefore, chelatable EDDHA to be prepared in the disclosure of the present application means A and B excluding C.

As an existing method for synthesizing EDDHA, a reaction material including phenol, ethylenediamine, glyoxylic acid and sodium hydroxide is reacted at 70° C. to 75° C., and then precipitated for 72 hours or longer at room temperature to obtain a product.

However, the reaction requires a precipitation time of at least three days (72 hours) to obtain the product after the synthesis, and due to the relatively long process time, the product produced using the method includes a large number of residues and other byproducts, and other organic solvents are additionally used to purify the product, causing a problem of generating additional waste liquid.

Accordingly, in order to resolve the above-described problems, the inventors of the present disclosure have identified that high-purity chelatable EDDHA may be prepared on an economical and industrial scale by synthesizing EDDHA with a more superior yield through synthesizing a mixture of substituted or unsubstituted phenol, ethylenediamine, glyoxylic acid and sodium hydroxide while raising the temperature to a predetermined temperature and thereby applying sufficient heat energy required for the synthesis reaction, and increasing a collection rate of the product through precipitation with heating at a certain temperature, and have completed the present disclosure.

According to the present disclosure, EDDHA may be prepared with an excellent yield when mixing substituted or unsubstituted phenol, ethylenediamine, glyoxylic acid and sodium hydroxide (step S1), and then reacting the mixture at 75° C. to 80° C. to synthesize a product (step S2) compared to when performing a reaction at 70° C. to 75° C., as known in the related art.

This is considered to be an effect resulting from the fact that sufficient heat energy required for the reaction may be obtained as the reaction proceeds at a temperature higher than 70° C. to 75° C. known in the related art by about 5° C. or higher on average, and moisture included in the reaction material evaporates with the high reaction temperature, reducing the moisture content.

When the reaction temperature is higher than 80° C., heat energy required for the reaction is sufficient, however, the heat is applied more than necessary, which may decompose the synthesized compound by heat or increase other byproducts, reducing the yield of chelatable EDDHA intended to be synthesized.

Herein, step (S2) of synthesizing a product by reacting the mixture at 75° C. to 80° C. may be performed for about 2 hours to 4 hours, preferably about 2 hours and 30 minutes to 3 hours and 30 minutes, and more preferably about 3 hours.

When the reaction time is less than 2 hours, the time taken for synthesizing the product is not sufficient, reducing the reaction yield, and the reaction time of longer than 4 hours is not preferred since EDDHA is polymerized and changes into an unusable byproduct.

Herein, the concentration of the sodium hydroxide may be from 35 wt % to 50 wt %, and preferably from 40 wt % to 50 wt %.

In the present application, 35 wt % to 50 wt % of an aqueous sodium hydroxide solution is used, whereas 30 wt % of an aqueous sodium hydroxide solution is used in the related art. Specifically, sodium hydroxide may not be present in the concentration of 30 wt % or greater at room temperature, however, 50 wt % of an aqueous sodium hydroxide solution may be prepared without additional energy by the heat generated during the process of mixing sodium hydroxide and water, and synthesis efficiency may be improved therethrough.

Meanwhile, due to the addition of sodium hydroxide, the mixture obtained by mixing substituted or unsubstituted phenol, ethylenediamine, glyoxylic acid and sodium hydroxide may undergo the reaction under a pH of about 8 to 10.

By the synthesis reaction in step (S2), chelatable EDDHA including o,o-EDDHA and op-EDDHA is synthesized and present while being dissolved in a solvent, and this may be heated to 55° C. to 65° C. to collect the product (step S3).

In the related art, a reaction material is left unattended for 72 hours or longer at room temperature in order to remove an organic solvent during the process of separating a product to obtain a precipitate, and then the precipitate is filtered to obtain a product. However, this is uneconomical since it takes a long time to obtain a product, and it is difficult to produce high-purity EDDHA on an industrial scale since the organic solvent is not sufficiently removed during the process of separating the product.

The inventors of the present disclosure have identified that, when heating the product synthesized in step (S2) for a certain period of time at 55° C. to 65° C., the time taken for collecting the product may be noticeably shortened.

In other words, when the product is collected at about 55° C. to 65° C. and preferably about 60° C., the synthesized product is not decomposed, and therefore, the organic solvent may be removed at a high speed while having thermal stability, and during this process, byproducts and contaminants are removed as well, and a high-purity product may be collected.

For example, dichloromethane (CH₂Cl₂) may be used as the organic solvent in the process of collecting the synthesized product through layer separation, and residual dichloromethane may be removed in a boiled form when heated to 55° C. to 65° C. Unreacted phenol may also be additionally removed with increased volatility when heated to 55° C. to 65° C.

Herein, step (S3) of collecting the product by heating the product to 55° C. to 65° C. may be performed for about 30 minutes to 2 hours, preferably about 1 hour to 1 hour and 30 minutes, and more preferably about 1 hour.

When the collecting time is less than 30 minutes, the organic solvent is not sufficiently removed, failing to sufficiently obtain the product, and the collecting time of longer than 2 hours is not preferred since the product may be decomposed.

The method for preparing chelatable EDDHA of the present disclosure includes the step of washing the collected product using a cleaning solution including deionized water (S4).

Through step (S4), metals and synthetic residues remaining in the product are additionally removed, and chelatable EDDHA with higher purity may be prepared.

Herein, the cleaning solution may further include an additive selected from the group consisting of an organic solvent including acetone, an organic base including NH₄OH, an inorganic acid including HNO₃ and HCl, and combinations thereof.

The additive performs a role of removing metal contaminants, and may be present in an ion form.

Specifically, the deionized water and the additive selected from the group consisting of an organic solvent including acetone, an organic base including NH₄OH, an inorganic acid including HNO₃ and HCl, and combinations thereof may be mixed in a weight ratio of 5:1 to 20:1, preferably 7:1 to 15:1, and more preferably 8:1 to 12:1.

According to the preparation method of the present disclosure, the chelatable EDDHA product may be prepared with an excellent yield of 18% or greater, preferably 19% or greater and more preferably 20% or greater, and may also be prepared with high purity of 90% or greater, preferably 95% or greater and more preferably 97% or greater.

According to the preparation method of the present disclosure, byproducts are reduced by synthesizing the mixture of substituted or unsubstituted phenol, ethylenediamine, glyoxylic acid and sodium hydroxide while raising the temperature to a predetermined temperature and thereby applying sufficient heat energy required for the synthesis reaction, and byproducts and contaminants are removed as well through precipitation by heating at a certain temperature, and as a result, a high-purity product may be prepared.

For example, the amounts of Al and Fe included in the product prepared using the preparation method may each be 5 ppm or less, preferably 3 ppm or less, and more preferably 2 ppm or less.

In addition, the amounts of K and Ca included in the product prepared using the preparation method may each be 5 ppm or less, preferably 4 ppm or less, and more preferably 3 ppm or less.

The amount of Na included in the product prepared using the preparation method may be 40 ppm or less, preferably 35 ppm or less, and more preferably 30 ppm or less.

As sodium hydroxide is used as the reaction material in the process for synthesizing EDDHA, Na metal may be included in the prepared EDDHA as an impurity, and it is preferred to synthesize EDDHA using the preparation method of the present application since the amount of Na included in the product may be reduced to 40 ppm or less.

In other words, chelatable EDDHA prepared using the preparation method has a low metal content in the product, and may be used for various purposes in a semiconductor process.

Hereinafter, embodiments of the present disclosure will be described in more detail with reference to the following examples. However, the following examples are just for illustrating embodiments of the present disclosure, and the present disclosure is not limited by the following examples.

### (1) Example 1

After dissolving phenol (100.0 g, 1.063 mol) in a two-neck round bottom flask equipped with a reflux condenser and a magnetic stir bar at 40° C. to 45° C., dry ethylenediamine (2.46 g, 40.9 mmol) was added thereto, and the mixture was stirred for 10 minutes.

To the stirred mixture, 50% NaOH (3.33 g, 40.9 mmol) and 50% glyoxylic acid (12.12 g, 81.8 mmol) were slowly added dropwise.

The mixture was heated for 3 hours at 75° C. to 80° C. to synthesize a product. After the temperature reaches room temperature, water (120 mL) and CH₂Cl₂ (240 mL) were added, and a produced mixture was stirred for 10 minutes.

After that, the result was heated for 1 hour at 60° C. in a constant temperature water bath, and then a precipitated product was filtered and collected.

Deionized water and the synthesized product were mixed in a weight ratio of 10:1, mixed for 20 minutes, separated, washed, and dried at 60° C. to obtain a mixture (3.45 g) of o,o-EDDHA and o,p-EDDHA.

### (2) Example 2

A mixture (3.45 g) of o,o-EDDHA and o,p-EDDHA was obtained in the same manner as in Example 1, except that deionized water and acetone (Sigma Aldrich) were mixed in a weight ratio of 10:1 and used.

### (3) Example 3

A mixture (3 g) of o,o-EDDHA and op-EDDHA was obtained in the same manner as in Example 1, except that 33% NaOH (5 g, 40.9 mmol) was used as the sodium hydroxide.

### (4) Comparative Example 1

A mixture (2.4 g) of o,o-EDDHA and o,p-EDDHA was obtained in the same manner as in Example 1, except that the mixture of phenol, ethylenediamine, glyoxylic acid and sodium hydroxide was heated for 3 hours at 70° C. to 75° C.

### (5) Comparative Example 2

A mixture (2.1 g) of o,o-EDDHA and o,p-EDDHA was obtained in the same manner as in Example 1, except that the mixture of phenol, ethylenediamine, glyoxylic acid and sodium hydroxide was heated for 3 hours at 80° C. to 85° C.

### (6) Comparative Example 3

A mixture (1.95 g) of o,o-EDDHA and o,p-EDDHA was obtained in the same manner as in Example 1, except that the mixture of phenol, ethylenediamine, glyoxylic acid and sodium hydroxide was heated for 3 hours at 85° C. to 90° C.

### (7) Comparative Example 4

After dissolving phenol (100.0 g, 1.063 mol) in a two-neck round bottom flask equipped with a reflux condenser and a magnetic stir bar at 40° C. to 45° C., dry ethylenediamine (2.46 g, 40.9 mmol) was added thereto, and the mixture was stirred for 10 minutes.

To the stirred mixture, 33% NaOH (4.97 g, 40.9 mmol) and 50% glyoxylic acid (12.12 g, 81.8 mmol) were slowly added dropwise.

The mixture was heated for 3 hours at 75° C. to 80° C. to synthesize a product. After the temperature reaches room temperature, water (120 mL) and CH₂Cl₂ (240 mL) were added, and a produced mixture was stirred for 10 minutes.

After that, the result was left unattended for 72 hours (3 days) at room temperature, and then a precipitated product was filtered.

Deionized water and the synthesized product were mixed in a weight ratio of 10:1, mixed for 20 minutes, separated, washed, and dried at 60° C. to obtain a mixture (1.65 g) of o,o-EDDHA and o,p-EDDHA.

The room temperature refers to a natural temperature without heating or cooling, and generally indicates temperatures in a range of 20±5° C.

### (8) Comparative Example 5

A mixture (2.1 g) of o,o-EDDHA and op-EDDHA was obtained in the same manner as in Comparative Example 4, except that the produced mixture was left unattended for 168 hours (7 days) at room temperature.

Yield and purity of each product in the Examples and Comparative Examples were calculated and shown in the following Table 1. Purity of the product may be identified through NMR and IR analyses, and in the case of IR, the purity may be identified as a matching ratio (%) through peak matching.

TABLE 1

| | Condition of | Condition of Step | | |
|---|---|---|---|---|
| | Step (S2) | (S3) | Yield | Purity |
| Example 1 | Heating for 3 hours | Heating for 1 hour | 23% | 97% |
| | at 75~80° C. | at 60° C. | | |
| Example 2 | Heating for 3 hours | Heating for 1 hour | 23% | 97% |
| | at 75~80° C. | at 60° C. | | |
| Example 3 | Heating for 3 hours | Heating for 1 hour | 20% | 97% |
| | at 75~80° C. | at 60° C. | | |
| Comparative | Heating for 3 hours | Heating for 1 hour | 16% | 96% |
| Example 1 | at 70~75° C. | at 60° C. | | |
| Comparative | Heating for 3 hours | Heating for 1 hour | 14% | 96% |
| Example 2 | at 80~85° C. | at 60° C. | | |
| Comparative | Heating for 3 hours | Heating for 1 hour | 13% | 95% |
| Example 3 | at 85~90° C. | at60° C. | | |
| Comparative | Heating for 3 hours | Leaving unattended | 11% | 88% |
| Example 4 | at 75~80° C. | for 72 hours at | | |
| | | room temperature | | |
| Comparative | Heating for 3 hours | Leaving unattended | 14% | 92% |
| Example 5 | at 75~80° C. | for 168 hours at | | |
| | | room temperature | | |

Through Table 1, it was identified that, when preparing chelatable EDDHA according to the preparation method of the present disclosure, high-purity EDDHA was able to be synthesized with excellent yield and purity by synthesizing the mixture of substituted or unsubstituted phenol, ethylenediamine, glyoxylic acid and sodium hydroxide while raising the temperature to a predetermined temperature and thereby applying sufficient heat energy required for the synthesis reaction, and EDDHA was able to be prepared at a high speed by increasing a collection rate of the product through precipitation with heating at a certain temperature.

### (9) Comparative Example 6

After dissolving phenol (100.0 g, 1.063 mol) in a two-neck round bottom flask equipped with a reflux condenser and a magnetic stir bar at 40° C. to 45° C., dry ethylenediamine (2.46 g, 40.9 mmol) was added thereto, and the mixture was stirred for 10 minutes.

To the stirred mixture, 50% NaOH (3.33 g, 40.9 mmol) and 50% glyoxylic acid (12.12 g, 81.8 mmol) were slowly added dropwise.

The mixture was heated for 3 hours at 75° C. to 80° C. to synthesize a product. After the temperature reaches room temperature, water (120 mL) and CH₂Cl₂ (240 mL) were added, and a produced mixture was stirred for 10 minutes.

After that, the result was heated for 1 hour at 60° C. in a constant temperature water bath, and then a precipitated product was filtered to obtain a mixture (3.45 g) of o,o-EDDHA and o,p-EDDHA.

### (10) Comparative Example 7

After dissolving phenol (100.0 g, 1.063 mol) in a two-neck round bottom flask equipped with a reflux condenser and a magnetic stir bar at 40° C. to 45° C., dry ethylenediamine (2.46 g, 40.9 mmol) was added thereto, and the mixture was stirred for 10 minutes.

To the stirred mixture, 50% NaOH (3.33 g, 40.9 mmol) and 50% glyoxylic acid (12.12 g, 81.8 mmol) were slowly added dropwise.

The mixture was heated for 3 hours at 70° C. to 75° C. to synthesize a product. After the temperature reaches room temperature, water (120 mL) and CH₂Cl₂ (240 mL) were added, and a produced mixture was stirred for 10 minutes.

After that, the result was left unattended for 72 hours (3 days) at room temperature, and then a precipitated product was filtered to obtain a mixture (1.6 g) of o,o-EDDHA and o,p-EDDHA.

The metal content in the product obtained in each of Example 1, Comparative Example 6 and Comparative Example 7 was analyzed and shown in the following Table 2.

The metal content in the EDDHA was analyzed after wet decomposition using the following method. Specifically, EDDHA (0.1 g) was decomposed with 70% nitric acid (10 ml) using Anton parr MULTIWAVE 5000 60 HZ PACKAGE 24HVT80, followed by collecting with 3% nitric acid, and then the metal content was analyzed using ICP-MS 8900.

TABLE 2

| | | Comparative | Comparative |
|---|---|---|---|
| Type of Metal (ppm) | Example 1 | Example 6 | Example 7 |
| Li | 0.00 | 0.10 | 0.03 |
| Be | 0.00 | 0.01 | 0.02 |
| B | 0.22 | 0.09 | 1.81 |
| Na | 29.35 | 2342.23 | 44.40 |
| Mg | 0.07 | 4.99 | 0.92 |
| Al | 1.31 | 4.22 | 6.24 |
| K | 0.55 | 8.79 | 18.19 |
| Ca | 2.48 | 19.09 | 18.56 |
| Cr | 0.02 | 0.08 | 0.09 |
| Mn | 0.00 | 0.13 | 0.04 |
| Fe | 0.26 | 3.70 | 1.87 |
| Co | 0.02 | 0.03 | 0.03 |
| Ni | 0.02 | 0.79 | 0.52 |
| Cu | 0.00 | 0.32 | 0.32 |
| Zn | 0.41 | 1.31 | 0.47 |
| As | 0.00 | 0.00 | 0.00 |
| Y | 0.00 | 0.00 | 0.00 |
| Sn | 0.00 | 0.00 | 0.06 |
| Tl | 0.00 | 0.00 | 0.00 |
| Pb | 0.03 | 0.13 | 0.22 |

Through Table 2, it was identified that, when preparing chelatable EDDHA according to the preparation method of the present disclosure, metals and synthetic residues present in the product were additionally removed, and chelatable EDDHA with higher purity was able to be prepared.

1. A method for preparing chelatable EDDHA, the method comprising the steps of:
mixing phenol, ethylenediamine, glyoxylic acid and sodium hydroxide (S1);
synthesizing a product by reacting the mixture at 75° C. to 80° C. (S2);
collecting the product by heating the product to 55° C. to 65° C. (S3); and
washing the collected product using a cleaning solution including deionized water (S4).

2. The method of claim 1, wherein, in step (S2), the mixture is reacted for 2 hours to 4 hours at 75° C. to 80° C. to synthesize a product.

3. The method of claim 1, wherein, in step (S3), the product is heated for 30 minutes to 2 hours at 55° C. to 65° C. to collect the product.

4. The method of claim 1, wherein a concentration of the sodium hydroxide is from 35 wt % to 50 wt %.

5. The method of claim 1, wherein a synthesis yield of the product prepared using the method is 18% or greater.

6. The method of claim 1, wherein amounts of Al and Fe included in the product prepared using the method are each 5 ppm or less.

7. The method of claim 1, wherein amounts of K and Ca included in the product prepared using the method are each 5 ppm or less.

8. The method of claim 1, wherein an amount of Na included in the product prepared using the method is 40 ppm or less.

9. The method of claim 1, wherein the cleaning solution further includes an additive selected from the group consisting of an organic solvent including acetone, an organic base including NH₄OH, an inorganic acid including HNO₃ and HCl, and combinations thereof.

10. The method of claim 9, wherein the cleaning solution includes the deionized water and the additive in a weight ratio of 5:1 to 20:1.
